(19) United States
(12) Patent Application Publication
(10) Pub. No.: US 2021/0284707 A1
(43) Pub. Date: Sep. 16, 2021

(54) GLUCOSE-RESPONSIVE INSULIN ANALOGS AND METHODS OF USE THEREOF

(71) Applicant: AMIDEBIO, LLC, Louisville, CO (US)

(72) Inventors: Michael H.B. STOWELL, Boulder, CO (US); Mikhail PLAM, Boulder, CO (US)

(21) Appl. No.: 17/213,765

(22) Filed: Mar. 26, 2021

Related U.S. Application Data
(63) Continuation of application No. 16/499,932.
(60) Provisional application No. 62/480,859, filed on Apr. 3, 2017.

(51) Int. Cl. C07K 14/62

(52) U.S. Cl. CPC C07K 14/62; A61K 38/00

(57) ABSTRACT

The subject matter of this invention is directed towards insulin analogs that are stable and that are glucose-responsive. An insulin analog can be a single chain insulin (SCI). The binding affinity of insulin to the insulin receptor can be controlled by the glucose-bound conformation of insulin. The invention further discloses methods for the recombinant expression, purification, and refolding of an insulin analog.

### CROSS REFERENCE

This application is a continuation of U.S. application Ser. No. 16/499,932, filed Oct. 1, 2019, which is a U.S. National Phase Application under 35 U.S.C. § 371 of International Application No. PCT/US2018/025070, filed Mar. 29, 2018, which claims the benefit of U.S. Provisional Application No. 62/480,859, filed Apr. 3, 2017, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 7, 2018, is named 39538-712-601_SL.txt and is 14,182 bytes in size.

### BACKGROUND

Insulin is a hormone secreted by the pancreas and is involved in the metabolism of glucose. Glucose metabolism can result in the production of energy that is necessary for cellular processes. Abnormal insulin levels can result in dangerous blood glucose fluctuations, which can have severe cardiovascular implications. Patients with abnormal insulin levels closely monitor their blood glucose levels and use insulin injections to maintain normal insulin levels. However, minor insulin dosing changes can result in blood glucose fluctuations and therefore can require repeated monitoring and multiple injections per day. The present invention can provide a glucose-responsive treatment to regulate blood glucose levels in insulin disorders.

### SUMMARY

Provided herein are insulin analogs. An insulin analog can comprise a glucose binding site, wherein binding of a glucose molecule to the glucose binding site changes conformation of the insulin analog. In some embodiments, the insulin analog has a high transition barrier for changing from a T state to an R state in an absence of the glucose molecule binding to the glucose binding site. In some embodiments, the insulin analog has lower transition barrier for changing from a T state to an R state when the glucose molecule binds to the glucose binding site compared to an absence of the glucose molecule binding to the glucose binding site. In further embodiments, the insulin analog has a low affinity for an insulin receptor in the absence of the glucose molecule binding to the glucose binding site. In some embodiments, the insulin analog has a K_{D} of more than 0.1 nM, 0.5 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 25 nM, 30 nM, or 50 nM for an insulin receptor in an absence of the glucose molecule binding to the glucose binding site. In additional embodiments, the insulin analog has an increased affinity for an insulin receptor when the glucose molecule binds to the glucose binding site. In some embodiments, the insulin analog has K_{D} of less than 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2.0 nM, 2.5 nM, 3.0 nM, 5 nM, 10 nM, 15 nM, or 20 nM for an insulin receptor when the glucose molecule binds to the glucose binding site. In further embodiments, an affinity of the insulin analog for an insulin receptor increases by at least 2-fold, 3-fold, 5-fold, 10-fold, 100-fold, 200-fold, 300-fold, or 500-fold when the glucose molecule binds to the glucose binding site as compared to in an absence of the glucose molecule binding to the glucose binding site.

In some embodiments, the glucose binding site comprises a glucokinase sequence. In further embodiments, the glucokinase sequence comprises Thr 168, Lys 169, Asn 204, Asp 205, Asn 231, Glu 290, or any combination thereof.

In other embodiments, the glucose binding site comprises a sequence from the periplasmic glucose binding protein of Pseudomonas putida CSV86 (ppGBP). In further embodiments, the ppGBP sequence comprises His 379, Lys 92, And 301, Asp 303, Lys339, Trp 36, Glu 41, Trp 35, or any combination thereof.

In some embodiments, the glucose binding site has a has K_{D} from 1 mM to 10 mM for a glucose molecule.

In some embodiments, the insulin analog comprises a single chain insulin (SCI) of the formula: B chain -C′-A chain Formula (I) wherein the B chain and the A chain are modified human insulin chains; and wherein C′ covalently links the B chain and the A chain, and is a peptide of about 5 to 9 amino acids. In further embodiments, the C′ comprises the following sequence: Y-P-G-D-X (SEQ ID NO: 1), wherein X is any amino acid.

In some embodiments, the residues within C′ comprises the glucose binding site. In other embodiments, residues within the B chain comprise the glucose binding site. In still other embodiments, residue within the A chain comprises the glucose binding site. In still other embodiments, residues within the C′ and B chain comprise the glucose binding site. In some embodiments, residues within the C′ and A chain comprise the glucose binding site. In other embodiments, residues within the C′, A chain, and B chain comprise the glucose binding site.

A polynucleotide comprising a nucleic acid sequence that encodes the insulin analog according to any one of the previous embodiments. In some embodiments, a recombinant vector comprises the polynucleotide of the previous embodiment. In further embodiments, the recombinant vector is a plasmid. In some embodiments, the recombinant vector comprises an inducible promoter. In further embodiments, the inducible promoter is IPTG.

In some embodiments, the recombinant vector comprises a fusion tag. In further embodiments, the fusion tag is derived from oleosin. In other embodiments, the fusion tag encodes a chemically cleavable sequence.

A cell line transformed with the recombinant vector of any one of the previous embodiments.

A method for treating diabetes in a patient in need thereof comprising administering the insulin analog of any one of the previous embodiments to the patient. In some embodiments, the diabetes is type I diabetes.

A method for producing single chain insulin comprising the introduction of a recombinant vector of any one of the previous embodiments into an expression system, the expression of a protein comprising a fusion tag and single chain insulin, the cleavage of said fusion tag from said single chain insulin, and isolating said single chain insulin. A method for obtaining purified insulin analog comprising producing the insulin analog according to the previous embodiment and purifying the analog using affinity chromatography.

A therapeutic composition comprising the insulin analog of any one of the previous embodiments. In some embodiments, the therapeutic composition is formulated for oral delivery. In other embodiments, the therapeutic composition is formulated for injection.

In some embodiments, the therapeutic composition is formulated for once daily delivery, once weekly delivery, or once monthly delivery.

A method of identifying a glucose-responsive insulin analog of any one of previous embodiments, the method comprising: i) identifying an insulin analog with a low affinity for insulin receptor; ii) introducing at least one mutation into the insulin analog, wherein the mutation allows a glucose molecule to bind to a glucose binding site on the insulin analog or alters binding of a glucose molecule to the glucose binding site on the insulin analog; iii) using normal mode analysis to identify the conformation of the insulin analog when a glucose molecule is bound the glucose binding site and in an absence of a glucose molecule binding to the glucose binding site; iv) further testing for affinity of the insulin analog to the insulin receptor when a glucose molecule is bound the glucose binding site and in the absence of a glucose molecule binding to the glucose binding site; and v) determining the insulin analog is a glucose-response insulin analog if the affinity of the insulin analog to the insulin receptor is at least 2 fold higher when a glucose molecule is bound to the glucose binding site than in the absence of a glucose molecule binding to the glucose binding site.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention can be obtained by reference to the following detailed description that sets forth illustrative embodiments, in which the principles of the invention are utilized, and the accompanying drawings of which:

FIG. 1 illustrates a diagram of Normal Mode Analysis (NMA) of insulin and insulin containing the Wakayama mutation (ValA3Leu) depicting force produced at each residue of the B chain. The Wakayama ValA3Leu insulin depicts a change in force at residue 8.

FIG. 2 illustrates exemplary models of a T structural conformation of the N terminus of a B-chain of insulin (left) and an R structural conformation of the N terminus of a B-chain of insulin (right).

FIG. 3 illustrates a diagram of NMA of single chain insulin analogs as compared with insulin, and depicts a decrease in relative binding affinity as a function of delta force.

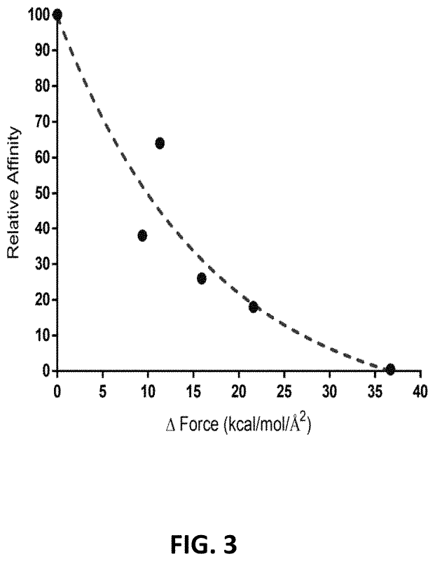

FIG. 4 illustrates an exemplary model of glucose binding to an insulin analog.

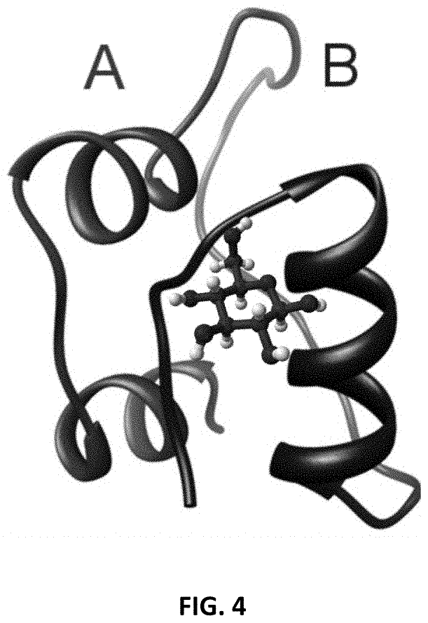

FIG. 5 illustrates a schematic of two chain human insulin comprising a native human insulin A chain and native human insulin B chain.

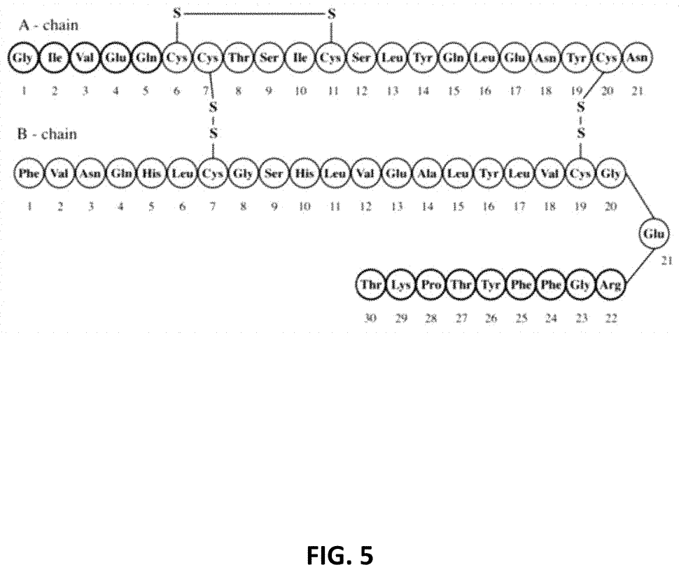

### DETAILED DESCRIPTION

The present disclosure provides compositions of glucose-responsive insulin analogs and methods of use thereof. These compositions and methods can be used for the treatment of insulin disorders, such as diabetes mellitus, insulinoma, metabolic syndrome, and polycystic ovary syndrome. Methods for identifying compositions of glucose-responsive insulin analogs are also provided herein. A composition can comprise an insulin analog. This insulin analog can change its structural conformation upon binding to glucose to regulate blood glucose levels.

Insulin is a hormone that regulates glucose metabolism and can be naturally secreted by pancreatic islet beta cells of the pancreas after detection of high levels of glucose in the blood stream. Insulin can initiate the metabolism of glucose by binding to insulin receptors on cellular membranes. The insulin receptor is encoded by a single gene, INSR, which is alternately spliced to result in an IR-A isoform and an IR-B isoform which contains an additional 12 amino acids. Both isoforms are cleaved to form α and β chains, with the addition 12 amino acids of the IR-B isoform remaining on the C terminal of the a chain. Two a chains and two β chains combine to form the transmembrane insulin receptor. The two a chains may be hetero- or homodimerized. Insulin can be released in the body after eating as blood levels of glucose rise rapidly. Upon binding of insulin to insulin receptors, glucose can be transported into the cell via glucose transporters and converted to glycogen or fat. This mechanism can reduce excessive circulating levels of glucose. In the absence of insulin, cells of the liver can convert glycogen back to glucose and release the glucose into the blood to maintain blood glucose levels.

In insulin disorders, insulin can be dysfunctional or may not be sufficiently produced by the pancreas. As a result, blood glucose may not be transported into cells to be metabolized. Under these circumstances, insulin can be provided to patients in need thereof. Because blood glucose levels need to be closely maintained within a healthy range, blood glucose levels need to be tightly regulated with frequent blood glucose monitoring and repeated insulin delivery. The ability to control insulin activity with glucose-responsive insulin introduces a way to regulate insulin activity based on innate glucose levels, and thus, to mimic innate glucose-regulated insulin activity.

### Insulin Peptide

As used herein, the term “peptide” can be any polymer comprising amino acids linked by peptide bonds. The term “peptide” can include polymers that can be assembled using a ribosome as well as polymers that can be assembled by enzymes (i.e., non-ribosomal peptides) and polymers that can be assembled synthetically. In various embodiments, the term “peptide” can be considered synonymous with “protein,” or “polypeptide.” In various embodiments, the term “peptide” can be limited to a polymer of greater than 50 amino acids, or alternatively, 50 or fewer amino acids. In various embodiments, the term “peptide” can include only amino acids as monomeric units for the polymer, while in various embodiments, the term “peptide” can include additional components and/or modifications to the amino acid backbone. For example, in various embodiments, the term “peptide” can be applied to a core polymer of amino acids as well as derivatives of the core polymer, such as core polymers with pendant polyethylene glycol groups or core polymers with amide groups at the amino or carboxy terminus of the amino acid chain.

Insulin can be composed of two peptide chains referred to as the A chain and the B chain. The A chain and B chain can be linked together by two disulfide bonds, and an additional disulfide can be formed within the A chain. In some embodiments, the A chain peptide is a peptide of 50 or fewer amino acids. In some embodiments, the A chain peptide is a peptide of 40 or fewer amino acids. In some embodiments, the A chain peptide is a peptide of 30 or fewer amino acids. In some embodiments, the B chain peptide is a peptide of 50 or fewer amino acids. In some embodiments, the B chain peptide is a peptide of 40 or fewer amino acids. In some embodiments, the B chain peptide is a peptide of 30 or fewer amino acids. In some embodiments, the B chain peptide is a peptide of 27-30 amino acids. In most species, the A chain can consist of 21 amino acids and the B chain can consist of 30 amino acids. Although the amino acid sequence of insulin can vary among species, certain segments of the molecule can be highly conserved, such as the positions of the three disulfide bonds, both ends of the A chain, and the C-terminal residues of the B chain. These similarities in the amino acid sequence of insulin can lead to a three dimensional conformation of insulin that can be very similar among species, and insulin from one animal can very likely be biologically active in other species. Indeed, pig insulin can be used to treat human patients. In some examples, the A chain comprises native human insulin A chain and the B chain comprises native human insulin B chain. In some examples, the A chain is native human insulin A chain and the B chain is native human insulin B chain. In some examples, the amino acid sequence of native human insulin A chain comprises GIVEQCCSICSLYQLENYCN (SEQ ID NO:8). In some examples, the amino acid sequence of native human insulin B chain comprises FVNQHLCGSHLVEALYLVCGERGFFYTPKT (SEQ ID NO:9). In some examples, the amino acid sequence of native human insulin A chain is provided as SEQ ID NO:8. In some examples, the amino acid sequence of native human insulin B chain is provided as SEQ ID NO:9.

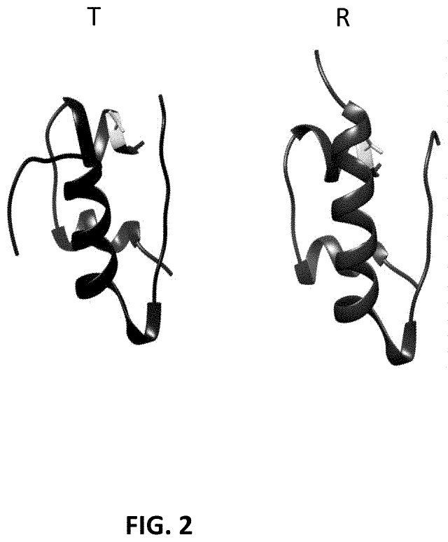

Insulin can exist in an active or inactive state, which can affect the kinetics and binding affinity of insulin to an insulin receptor. Insulin in its active state can initiate the metabolism of glucose by binding to insulin receptors on cellular membranes. In contrast, insulin in its inactive state can have decreased binding affinity for insulin receptors, resulting in decreased cellular glucose uptake. The active state or inactive state of insulin can be dictated by its structural conformation. For example, the N-terminus of the B chain of insulin can exist in two different conformational states: the T state, which can be characterized as comprising an extended N-terminus, and the R state, which can be characterized as comprising a helical N-terminus. An exemplary model of the insulin B chain in a T state and an exemplary model of the insulin B chain in an R state are shown in FIG. 2. The T to R conversion is believed to be important for binding of insulin to the insulin receptor.

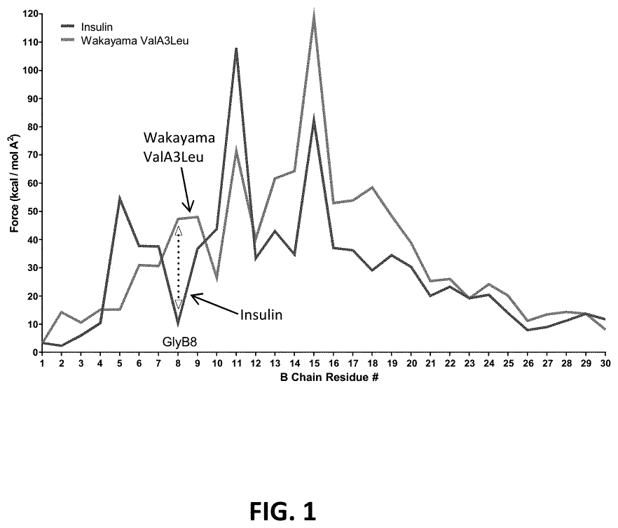

Furthermore, mutations in insulin can affect the conformational state of insulin by changing the required transition energy for insulin to undergo a conformational change, and therefore, can also change the affinity of insulin for the insulin receptor. For example, the Wakayama mutation ValA3Leu, a naturally occurring mutation in insulin, can create a high transition state barrier for the conformational change from the T state to the R state. As shown in FIG. 1, the mutated glycine residue at position B8 can create a higher transition state barrier for the conformational change from the T state to the R state as compared to wildtype insulin, thus resulting in an insulin with 500-fold lower affinity for the insulin receptor compared to wildtype insulin. Therefore, the transition state barrier and structural conformation unexpectedly correlate with a change in affinity of the insulin for the insulin receptor, which can be found to occur even with only minor perturbations in the insulin sequence. In patients, the Wakayama mutation ValA3Leu can have impaired folding and disulfide bond formation of pre-insulin, and permanent neonatal-onset diabetes mellitus.

### Glucose-Responsive Single Chain Insulin Analogs

Single chain insulin (SCI) can encompass a group of structurally-related proteins wherein the A chain and B chain can be covalently linked by a polypeptide linker. SCI can have greater insulin receptor binding activity and/or glucose uptake activity compared to proinsulin, and lesser insulin receptor binding activity and glucose uptake activity compared to insulin. Modification of the linker can provide substantial thermodynamic stability in various embodiments.

In some examples, the insulin analog comprises a single chain insulin (SCI) of the formula:

B chain-C′-A chain Formula (I)

wherein the B chain and the A chain are respectively native or modified human insulin B chain and A chain, wherein C′ is a polypeptide linker which covalently links the B chain and the A chain. A polypeptide linker can connect the C-terminus of the B chain to the N-terminus of the A chain. The linker can be any length so long as the linker provides the structural conformation necessary for SCI to have a glucose uptake and insulin receptor binding effect. For example, the linker can about 3-9, for example about 4-9 or about 5-9 amino acids long. In some embodiments, the linker is about 9 amino acids long. In some examples, the linker is about 8 amino acids long. In some examples, the linker is about 7 amino acids long. In some examples, the linker is about 6 amino acids long. In some examples, the linker is about 5 amino acids long. In some examples, the linker is about 4 amino acids long.

In some examples, the linker is be Y-P-G-D-X (SEQ ID NO:1) wherein X is any amino acid. However, it should be understood that many variations of this sequence are possible such as in the length (both addition and deletion) and substitutions of amino acids without substantially compromising the effectiveness of the produced SCI in glucose uptake and insulin receptor binding activities. For example, several different amino acid residues may be added or taken off at either end of the linker without substantially decreasing the activity of the produced SCI. In addition, the amino acid Gly can be replaced with any amino acid residue. It is also to be understood that the insulin A chain and B chain can be modified in various embodiments to enhance chemical stability.

In some embodiments a polypeptide linker connects the C-terminus of the B chain to the N-terminus of the A chain. The linker may be of any length so long as the linker provides the structural conformation necessary for SCI to have a glucose uptake and insulin receptor binding effect.

In some embodiments, the insulin analog of the present invention comprises a motif in the polypeptide linker region comprising Y₁Y₂Y₃Y₄Y₅Y₆Y₇ (SEQ ID NO:11), wherein Y₁ is R or absent, Y₂ is R or absent, Y₃ is Y or V, Y₄ is P or absent, Y₅ is G or D, Y₆ is D or G, and Y₇ is V or K. In some embodiments, the insulin analog of the present invention comprises a motif in the polypeptide linker region comprising Y₁Y₂Y₃Y₄GDY₇ (SEQ ID NO:12), wherein Y₁ is R or absent, Y₂ is R or absent, Y₃ is Y or V, Y₄ is P or absent, and Y₇ is V or K. In some embodiments, the insulin analog of the present invention comprises a motif in the polypeptide linker region comprising Y₁Y₂Y₃Y₄DGY₇ (SEQ ID NO:13), wherein Y₁ is R or absent, Y₂ is R or absent, Y₃ is Y or V, Y₄ is P or absent, and Y₇ is V or K.

In some embodiments, the insulin analog of the present invention comprises a motif in the polypeptide linker region comprising Y₁Y₂Y₃Y₄DGV (SEQ ID NO:14), wherein Y₁ is R or absent, Y₂ is R or absent, Y₃ is Y or V, and Y₄ is P or absent. In some embodiments, the insulin analog of the present invention comprises a motif in the polypeptide linker region comprising Y₁Y₂Y₃Y₄DGK (SEQ ID NO:15), wherein Y₁ is R or absent, Y₂ is R or absent, Y₃ is Y or V, and Y₄ is P or absent.

In some embodiments, the insulin analog of the present invention comprises a motif in the polypeptide linker region comprising RRYPGDV (SEQ ID NO:16). In some embodiments, the insulin analog of the present invention comprises a motif in the polypeptide linker region comprising RRYPDGV (SEQ ID NO:17). In some embodiments, the insulin analog of the present invention comprises a motif in the polypeptide linker region comprising RRVDGV (SEQ ID NO:18). In some embodiments, the insulin analog of the present invention comprises a motif in the polypeptide linker region comprising RYPDGV (SEQ ID NO:19). In some embodiments, the insulin analog of the present invention comprises a motif in the polypeptide linker region comprising RVDGV (SEQ ID NO:20). In some embodiments, the insulin analog of the present invention comprises a motif in the polypeptide linker region comprising VDGK (SEQ ID NO:21). In some embodiments, the insulin analog of the present invention comprises a motif in the polypeptide linker region comprising YDGK (SEQ ID NO:22).

In some embodiments, the insulin analog of the present invention comprises a motif comprising X₁X₂X₃QHLCGSHLVEALYLVCGERGFFYTPKTY₁Y₂Y₃Y₄Y₅Y₆Y₇GIVEQCCTSICSLYQLENY CZ₁ (SEQ ID NO:10), wherein X₁ is F or absent, X₂ is V or absent, X₃ is N, A, K, D, or absent, Y₁ is R or absent, Y₂ is R or absent, Y₃ is Y or V, Y₄ is P or absent, Y₅ is G or D, Y₆ is D or G, and Y₇ is V or K, Z₁ is N, G, A, D, or Q.

In some embodiments, the insulin of the present invention comprises a motif comprising X₁X₂X₃QHLCGSHLVEALYLVCGERGFFYTPKTY₁Y₂Y₃Y₄Y₅Y₆Y₇GIVEQCCTSICSLYQLENY CZ₁, where X₁ represents F or Des, X₂ represents V or Des, X₃ represents N, A, K, D or Des, Y₁ represents R or Des, Y₂, represents R or Des, Y₃ represents Y, R or Des, Y₄ represents, P, V, or Y, Y₅ represents, G or D, Y₆ represents D or G, Y₇ represents V or K, and Z₁ represents N, G, A, D, Q. Des corresponds to the lack of that particular amino acid in the single chain insulin (e.g. Des 12 refers to the deletion of residue twelve.

“Insulin analog” as used herein can refer to a single chain insulin (SCI) wherein one or more amino acid residues have been replaced by another amino acid residue, wherein one or more amino acid residues have been deleted, or wherein one or more amino acid residues have been added to the A chain or B chain at the N-terminal or at the C-terminal. The analog used herein can control the level of glucose in the blood. This analog can control the level of glucose in the blood with different pharmacokinetics than wildtype or naturally occurring insulin. “Insulin derivative” as used herein can be a wildtype or naturally occurring insulin or insulin analog, which has been chemically modified. Examples of chemical modifications can include the addition of a side chain to one or more positions of the insulin A chain or B chain or by chemically modifying amino acid residues such as by oxidation, reduction, or acetylation.

Insulin analogs can have a high transition state barrier for the conformational change from the T state to the R state as compared to wildtype or naturally occurring insulin. In some embodiments, the insulin analog can comprise a glucose binding site. The glucose binding site can be cryptic glucose binding site. The binding of a glucose molecule to the glucose binding site can lead to an allosteric conformational change of the insulin analog. This change in conformation can alter the binding affinity of the insulin to the insulin receptor. For example, an insulin analog with a glucose binding site can have a low affinity for an insulin receptor in the absence of a glucose molecule binding to the glucose binding site. However, if a glucose molecule is bound, this binding can cause an allosteric conformational change that can lead to a lower transition state barrier for the conformational change from the T state to the R state. As a result, the binding affinity of the insulin analog to the insulin receptor can be increased. Therefore, this change in the binding affinity of the insulin analog for an insulin receptor can be dependent on whether a glucose molecule is bound to the glucose binding site. Stated another way, when a glucose molecule is not bound to the insulin analog, the insulin analog can have a high transition state barrier and thus the insulin analog maintains a low binding affinity for an insulin receptor. In contrast, when a glucose molecule binds to the glucose binding site of the insulin analog, the transition state barrier can be decreased, which can allow the insulin analog to undergo the conformational change from the T state to the R state, resulting in a higher binding affinity for an insulin receptor. As another example, when a glucose molecule binds to the insulin analog, the insulin analog can have an increased binding affinity for an insulin receptor as compared to the insulin analog when a glucose molecule is not bound to the glucose binding site. This can allow for the insulin analog to be active (i.e., high affinity for an insulin receptor) in the presence of high blood glucose levels, and conversely, the insulin analog to be inactive (i.e., low affinity for an insulin receptor) when blood glucose levels are low. Hence, an insulin analog with these characteristics can be referred to as a glucose-responsive insulin analog.

In some instances, an insulin analog can have an affinity (K_{D}) of more than 0.1 nM, 0.5 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 25 nM, 30 nM, 50 nM or higher for an insulin receptor when a glucose molecule is not bound to the glucose binding site of the insulin analog. In other instances, an insulin analog can have an affinity (K_{D}) of more than 1 nM for an insulin receptor when a glucose molecule is not bound to the glucose binding site of the insulin analog. An insulin analog can have an affinity (K_{D}) of more than 2 nM for an insulin receptor when a glucose molecule is not bound to the glucose binding site of the insulin analog. An insulin analog can have an affinity (K_{D}) of more than 5 nM for an insulin receptor when a glucose molecule is not bound to the glucose binding site of the insulin analog. An insulin analog can have an affinity (K_{D}) of more than 8 nM for an insulin receptor when a glucose molecule is not bound to the glucose binding site of the insulin analog. An insulin analog can have an affinity (K_{D}) of more than 10 nM for an insulin receptor when a glucose molecule is not bound to the glucose binding site of the insulin analog. An insulin analog can have an affinity (K_{D}) of more than 12 nM for an insulin receptor when a glucose molecule is not bound to the glucose binding site of the insulin analog. An insulin analog can have an affinity (K_{D}) of about 5 nM for an insulin receptor when a glucose molecule is not bound to the glucose binding site of the insulin analog. An insulin analog can have an affinity (K_{D}) of about 10 nM for an insulin receptor when a glucose molecule is not bound to the glucose binding site of the insulin analog. In some instances, an insulin analog can have an affinity (K_{D}) of about 0.1 nM to about 50 nM, about 0.1 nM to about 10 nM, about 0.5 nM to about 10 nM, about 1 nM to about 10 nM, or about 5 nM to about 10 nM when a glucose molecule is not bound to the glucose binding site of the insulin analog.

The binding affinity of an insulin analog to an insulin receptor can be higher when a glucose molecule is bound to the glucose binding site of the insulin analog. The binding affinity of an insulin analogs to an insulin receptor can be about 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, 200-fold, 300-fold, 400-fold, 500-fold, or more than 500-fold higher when a glucose molecule is bound to the glucose binding site of the insulin analog than when a glucose molecule is not bound to the glucose binding site of the insulin analog. In some instances, the insulin analog can have an affinity (K_{D}) of less than 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2.0 nM, 2.5 nM, 3.0 nM, 5 nM, 10 nM, 15 nM, or 20 nM for an insulin receptor when a glucose molecule is bound to the glucose binding site of the insulin analog. The insulin analog can have an affinity (K_{D}) of less than 0.01 nM for an insulin receptor when a glucose molecule is bound to the glucose binding site of the insulin analog. The insulin analog can have an affinity (K_{D}) of less than 0.02 nM for an insulin receptor when a glucose molecule is bound to the glucose binding site of the insulin analog. The insulin analog can have an affinity (K_{D}) of less than 0.05 nM for an insulin receptor when a glucose molecule is bound to the glucose binding site of the insulin analog. The insulin analog can have an affinity (K_{D}) of less than 0.08 nM for an insulin receptor when a glucose molecule is bound to the glucose binding site of the insulin analog. The insulin analog can have an affinity (K_{D}) of less than 0.1 nM for an insulin receptor when a glucose molecule is bound to the glucose binding site of the insulin analog. The insulin analog can have an affinity (K_{D}) of about 0.05 nM for an insulin receptor when a glucose molecule is bound to the glucose binding site of the insulin analog. In some instances, an insulin analog can have an affinity of about 0.001 nM to about 25 nM, about 0.001 nM to about 1 nM, about 0.05 nM to about 1 nM, about 0.1 nM to about 1 nM, or about 0.5 nM to about 1 nM when a glucose molecule is bound to the glucose binding site of the insulin analog.

A glucose molecule can bind to the glucose binding site of the insulin analog through interactions with amino acids in the insulin A chain, the B chain, the linker or a combination thereof. The linker can be selected to allow for glucose binding. In some instances, the linker can comprise sequence from known glucose binding proteins such that they provide similar three dimensional structural arrangements of amino acid residues suitable for glucose binding. In some instances, the linker and surrounding residues can comprise sequence from the glucokinase protein, such as residues Thr 168, Lys 169, Asn 204, Asp 205, Asn 231, or Glu 290 of glucokinase. In some instances, the linker can comprise sequence from the periplasmic glucose binding protein of Pseudomonas putida CSV86 (ppGBP), such as residues His 379, Lys 92, Asn 301, Asp 303, Lys339, Trp 36, Glu 41, or Trp 35. In some instances, the linker can be structurally similar to the glucose binding site of Galectin from Toxoscaris Leonina comprised of residues His57, Asn70, Arg61, and Glu80. The residues of the insulin A chain and B chain can also be modified to facilitate glucose binding, for example, by altering a part of the sequence to provide a key glucose interacting residue as predicted by studies of glucose binding proteins. In some instances, the glucose binding site of the insulin analog can have an affinity (K_{D}) of less than 1 mM, 2 mM, 3 mM, 4 mM, 5 mM, 6 mM, 7 mM, 8 mM, 9 nM, or 10 mM for a glucose molecule. The glucose binding site of the insulin analog can have an affinity (K_{D}) of about 4 mM for a glucose molecule. The glucose binding site of the insulin analog can have an affinity (K_{D}) of about 5 mM for a glucose molecule. The glucose binding site of the insulin analog can have an affinity (K_{D}) of about 6 mM for a glucose molecule. The glucose binding site of the insulin analog can have an affinity (K_{D}) of about 7 mM for a glucose molecule. The glucose binding site of the insulin analog can have an affinity (K_{D}) of about 8 mM for a glucose molecule. The glucose binding site of the insulin analog can have an affinity (K_{D}) of about 1 mM to 10 mM for a glucose molecule. The glucose binding site of the insulin analog can have an affinity (K_{D}) of about 4 mM to 8 mM for a glucose molecule.

### Method of Identifying Glucose-Responsive Insulin Analogs

The conformational change of large proteins upon binding of a small molecule can be predicted using molecular dynamics simulation techniques. Proteins can be dynamic structures, and the fluctuations in protein structure can be important for determining their function. Normal mode analysis (NMA) is a molecular dynamic simulation technique to study the slow deformation of large molecules, e.g., large proteins, and insulin. The conformation can be related to the energy of the system. A normal mode can be a characteristic feature of the system and can occur when all parts of the system are moving sinusoidally with the same frequency and in phase. The energy of binding can be calculated at each normal mode of the bound molecules. The most probable binding affinity is at the normal mode that requires the least energy.

NMA is a type of multivariate analysis of the collective motions based on principal component analysis (PCA). Other PCA methods can be utilized for predicting the binding affinity of two molecules including essential dynamics analysis (EDA), singular value decomposition (SVD), and Monte Carlo (MC) approaches. These methods are based on determining the equilibrium dynamics of each defined system. The system is defined by the conformations of the molecules and the binding affinities at different regions.

Insulin analogs can be designed to be glucose-responsive. Some exemplary insulin analogs that can be used as the starting point for designing glucose-responsive insulin can be FVNQHLCGSHLVEALYLVCGERGFFYTPKTRRYPGDVGIVEQCCTSICSLYQLENYCG (SEQ ID NO: 2); FVNQHLCGSHLVEALYLVCGERGFFYTPKTRVDGVGIVEQCCTSICSLYQLENYCG (SEQ ID NO: 3); FVNQHLCGSHLVEALYLVCGERGFFYTPKTVDGKGIVEQCCTSICSLYQLENYCG (SEQ ID NO: 4); FVDQHLCGSHLVEALYLVCGERGFFYTPKTYDGKGIVEQCCTSICSLYQLENYCG (SEQ ID NO: 5); FVNQHLCGSHLVEALYLVCGERGFFYTPKTYDGKGIVEQCCTSICSLYQLENYCG (SEQ ID NO: 6); and FVGQHLCGSHLVEALYLVCGERGFFYTPKTYDGKGIVEQCCTSICSLYQLENYCG (SEQ ID NO: 7). For example, an insulin analog can be produced to have a lower T to R transition energy when a glucose molecule is bound to the glucose binding site of the insulin analog. As a first step, a single chain insulin analog with low affinity for the insulin receptor and a high T to R transition energy can be selected. Modification of the single chain insulin analog can then be designed to include a glucose binding site. The glucose binding site can be within the linker, or can comprise residues from the linker as well as the A chain and/or the B chain. Normal mode analysis can then be conducted in the presence or in the absence of a glucose molecule binding to the glucose binding site of the insulin analog to determine if glucose binding affects the transition barrier of the insulin analog. A modified insulin analog which has a lower T to R transition energy when glucose is bound to the glucose binding site of the insulin analog can be selected for further testing as a glucose-responsive insulin analog.

### Insulin Thermodynamic Stability and Fibrillation

Insulin molecules can form dimers in solution due to hydrogen-bonding between the C-termini of B chains. Additionally, in the presence of zinc ions, insulin dimers can associate into hexamers. These interactions can have important clinical ramifications. Monomers and dimers may readily diffuse into blood, whereas hexamers may diffuse very poorly. Hence, absorption of insulin preparations containing a high proportion of hexamers may be delayed and slow.

An important characteristic of formulated insulin which significantly can limit both storage and in use stability can be a propensity to undergo fibrillation, an irreversible non-covalent polymerization process which causes the insulin molecules to aggregate and form insoluble linear fibrils. The process can be most favored under acidic pH and elevated temperature conditions, and can be exacerbated by agitation and by the presence of excess zinc ions. The consequences of increased insulin fibril content can include a gradual attenuation of the pharmacological potency of the insulin preparation as well as the possibility of increased immunogenicity. Mechanistically, this complex phenomenon is thought to be initiated through displacement or “unfolding” of the B-chain C-terminus and the resulting exposure of the non-polar residues IleA2, ValA3, LeuB11 and LeuB15 which then form a hydrophobic interface facilitating the fibrillation event.

The tendency towards fibrillation can be effectively minimized in cases where C-terminal residues of the B-chain are restricted by their conformation by tethering to the N-terminus of the A-chain thereby forming a continuous single chain insulin. For example, proinsulin can be significantly less prone to fibrillation than insulin and cleavage of its C-peptide readily restores the fibrillation tendency. Other single chain insulin peptides with shorter C-peptides have been shown to resist fibrillation. Conversely, C-terminally truncated analogs such des(B26-30) insulin are known to fibrillate even more rapidly than native insulin, supporting the view that the B-chain C-terminus plays a critical role in the fibrillation process.

Long term chemical stability of insulin can be affected by pH and temperature. In addition, there are a number of secondary factors, which can influence long-term stability. These include the type of crystal structure, the presence of bacteriostatic agents (phenol, m-cresol), buffering reagents (phosphate, TRIS), isotonicity additives (glucose, glycerol, NaCl), and substances added to protract insulin's time of action profile (protamine sulfate and Zn⁺⁺).

With regard to chemical degradation patterns of human insulin, while any of the six amide containing side chains in insulin (GlnA5, GlnA15, AsnA18, AsnA21, AsnB3, GlnB5) can undergo deamidation, in the context of commercial formulations only the asparagines at A21 and B3 are of specific concern. Of the two, AsnA21 is the more labile site with up to 20-30% deamidation noted after one year in acidic formulations. Mechanistically, the AsnA21 degradation can proceed via an aspartimide intermediate to give either the aspartic acid derivative, or through reaction with another insulin molecule, a covalent insulin dimer or higher order molecular weight transformation product. AsnB3 hydrolytic decomposition can occur under neutral conditions and can result in formation of AspB3 and isoAspB3 in roughly equal proportions. In certain crystalline zinc formulations, the A-chain can also undergo backbone cleavage between ThrA8 and SerA9. In some embodiments, the A chain of a single chain insulin of the present invention comprises SEQ ID NO:8. In some embodiments, the A chain of a single chain insulin of the present invention comprises SEQ ID NO:8, with one or more mutations at amino acid residues selected from the group consisting of Gln5, Gln15, Asn18, and Asn21. In some embodiments, the B chain of a single chain insulin of the present invention comprises SEQ ID NO:9. In some embodiments, the B chain of a single chain insulin of the present invention comprises SEQ ID NO:9, with one or more mutations at amino acid residues selected from the group consisting of Asn3 and Gln5. In some embodiments, the A chain comprises SEQ ID NO:8 and the B chain comprises SEQ ID NO:9. In some embodiments, the A chain comprises SEQ ID NO:8 with one or more mutations at amino acid residues selected from the group consisting of Gln5, Gln15, Asn18, and Asn21 and the B chain comprises SEQ ID NO:9 with one or more mutations at amino acid residues selected from the group consisting of Asn3 and Gln5.

In some embodiments, the chemical stability of the single chain insulin disclosed herein is increased at least about 1.1 fold, about 1.2 fold, about 1.3 fold, about 1.4 fold, about 1.5 fold, about 1.6 fold, about 1.7 fold, about 1.8 fold, about 1.9 fold, about 2 fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 200-fold, about 300-fold, about 400-fold or about 500-fold compared to native human insulin.

In some embodiments, the propensity of a single chain insulin described herein to undergo fibrillation is reduced about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% compared to native human insulin.

### Treatment of Disease

A composition comprising SCI and any variant disclosed herein can be used to treat a subject in need thereof. A subject in need thereof can have an insulin disease. Insulin diseases can be characterized by disturbed insulin production by the body. The body can be produce too low or excessively high levels of insulin depending on the condition thereof. Insulin diseases include, but are not limited to, diabetes mellitus, insulinoma, metabolic syndrome, and polycystic ovary syndrome.

### Diabetes Mellitus

Diabetes mellitus can refer to a disease with insulin deficiency or relative insulin deficiency. It can also include impaired glucose tolerance. Hyperglycemia or excessive blood sugar is a result of low insulin levels. Diabetes mellitus can include, but is not limited to, Type 1 diabetes or insulin-dependent diabetes mellitus (IDDM), Type 2 diabetes or non-insulin-dependent diabetes mellitus (NIDDM), gestational diabetes, and impaired glucose tolerance and prediabetes. Type 1 diabetes is also classified as an auto-immune disorder where the immune system attacks the insulin-producing beta cells of the pancreas. Type 2 diabetes is described in patients where the body fails to produce the required amount of insulin. Gestational diabetes is described in pregnant patients with insufficient insulin levels. Pregnant women require higher levels of insulin. Impaired glucose tolerance is a prediabetic condition where blood glucose is high but not high enough for a diagnosis of diabetes.

### Other Insulin Disorders

Insulinoma is a cancer of the pancreatic beta cells. Tumors in the pancreas lead to excess production of insulin and therefore result in hypoglycemia. Metabolic syndrome is a combination of clinical disorders where the underlying cause may be insulin resistance of type 2 diabetes. Polycystic ovary syndrome is a complex syndrome in women, who exhibit insulin resistance. Hyperinsulinemia is a disorder where levels of insulin are in excess.

### Pharmaceutical Composition

The therapeutic dose administered to a patient can vary between wide ranges, depending on the mode of administration, the formulation used, and the response desired.

The present disclosure provides a pharmaceutical composition comprising compositions disclosed herein and a pharmaceutically acceptable carrier. The carrier can be a liquid formulation, such as a buffered, isotonic, aqueous solution. Pharmaceutically acceptable carriers can also include excipients, such as diluents, carriers and the like, and additives, such as stabilizing agents, preservatives, solubilizing agents, buffers and the like.

Formulation excipients can include polyvinylpyrrolidone, gelatin, hydroxy cellulose, acacia, polyethylene glycol, mannitol, sodium chloride and sodium citrate. For injection or other liquid administration formulations, water containing at least one or more buffering constituents can be utilized and stabilizing agents, preservatives and solubilizing agents can also be employed. For solid administration formulations, any of a variety of thickening, filler, bulking and carrier additives can be employed, such as starches, sugars, fatty acids and the like. For topical administration formulations, any of a variety of creams, ointments, gels, lotions and the like can be employed. For most pharmaceutical formulations, non-active ingredients can constitute the greater part, by weight or volume, of the preparation. For pharmaceutical formulations, it is also contemplated that any of a variety of measured-release, slow-release or sustained-release formulations and additives can be employed so that the dosage can be formulated so as to affect the delivery of a composition disclosed herein over a period of time.

The pharmaceutical forms suitable for injectable use can include sterile aqueous solutions or dispersions and sterile powders for the extemporaneous preparation of sterile injectable solutions or dispersions. The form can be sterile and fluid to the extent that it can be administered by syringe. The form preferably is stable under the conditions of manufacture and storage and is preserved against the contaminating action of microorganisms such as bacteria and fungi. The carrier can be a solvent or dispersion medium containing, for example, water, ethanol, a polyol, for example glycerol, propylene glycol or liquid polyethylene glycol, suitable mixtures thereof, and vegetable oils.

Because of the ease of administration, tablets and capsules can represent an advantageous oral dosage unit form. If desired, tablets can be coated by standard aqueous or non-aqueous techniques. The tablets, pills, capsules, and the like can also contain a binder, such as gum tragacanth, acacia, corn starch or gelatin; excipients, such as dicalcium phosphate; a disintegrating agent, such as corn starch, potato starch or alginic acid; a lubricant, such as magnesium stearate; and a sweetening agent such as sucrose, lactose or saccharin. When a dosage unit form is a capsule, it can contain, in addition to materials of the above type, a liquid carrier such as fatty oil.

Compositions herein can also be administered parenterally. Solutions or suspensions can be prepared in water suitably mixed with a surfactant such as hydroxy-propylcellulose. Dispersions can also be prepared in glycerol, liquid polyethylene glycols and mixtures thereof in oils. These preparations can optionally contain a preservative to prevent the growth of microorganisms.

The pharmaceutical forms suitable for administration intranasally, intratracheally, or to the respiratory tract and lungs via inhalation can include an aerosol or solution for a nebulizer, or as a microtine powder for insufflation or inhalation (e.g., topically to the lung and/or airways), alone or in combination with one or more inert carriers or additional active pharmaceutical ingredients, and in the form of a solution, a suspension, an aerosol or a dry powder formulation. Compositions can be therapeutically applied by means of nasal administration, which refers to any form of intranasal administration. The compositions can be in an aqueous solution, such as a solution including saline, citrate or other common excipients or preservatives. The compositions can also be in a dry or powder formulation.

The pharmaceutical composition can also be delivered via topical drug delivery to the skin, conjunctiva, ear, mouth, and vaginal cavities, using formulations, including aqueous topical solutions, lotions, creams, ointments, gels, foams, powders, or pastes, either applied directly or in conjunction with methods and devices, such as iontophoresis, microneedles, or a transdermal patch.

If in an aqueous solution, the compositions can be appropriately buffered by means of saline, acetate, phosphate, citrate, acetate or other buffering agents, which can be at any physiologically acceptable pH, generally from about pH 4 to about pH 7. A combination of buffering agents can also be employed, such as phosphate buffered saline, a saline and acetate buffer, and the like. In the case of saline, a 0.9% saline solution can be employed. In the case of acetate, phosphate, citrate, and the like, a 50 mM solution can be employed. In addition to buffering agents, a suitable preservative can be employed, to prevent or limit bacteria and other microbial growth. One such preservative that can be employed is 0.05% benzalkonium chloride.

Compositions can be therapeutically administered by means of an injection of a sustained release formulation or via depot administration. Compositions herein can be formulated for administration by a deep intramuscular injection, such as in the gluteal or deltoid muscle, comprising a formulation with a polyethylene glycol, such as polyethylene glycol 3350, and optionally one or more additional excipients and preservatives, including but not limited to excipients such as salts, polysorbate 80, sodium hydroxide or hydrochloric acid to adjust pH, and the like. Alternatively other sustained release formulations can be employed for subcutaneous injection or local injection, e.g., intra-articular injection. Formulations can include one or more of nano/microspheres (such as compositions including PLGA polymers), liposomes, emulsions (such as water-in-oil emulsions), gels, zinc phosphate, insoluble salts, or suspensions in oil.

The solutions can also contain conventional, pharmaceutically acceptable preservatives, stabilizers, cosolvents and/or penetration enhancers as well as viscoelastic substances included in artificial tear preparations. Pharmaceutically acceptable preservatives include quaternary ammonium compounds such as benzalkonium chloride, benzoxonium chloride or the like; alkyl-mercury salts of thiosalicylic acid, such as thiomersal, phenylmercuric nitrate, phenylmercuric acetate or phenylmercuric borate; sodium perborate; sodium chlorite; parabens, such asmethylparaben or propylparaben; alcohols such as chlorobutanol, benzyl alcohol or phenyl ethanol; guanidine derivatives such as chlorohexidine or polyhexamethylene biguanide; sorbic acid; boric acid; or peroxide forming preservatives, or combinations of two or more of the foregoing. Pharmaceutically acceptable antioxidants and chelating agents can be used including various sulphites (such as sodium metabisulphite, sodium thiosulphate, sodium bisulfite, or sodium sulfite), a-tocopherol, ascorbic acid, acetylcysteine, 8-hydroxyquinolome, antipyrine, butylated hydroxyanisole or butylated hydroxytoluene, EDTA, and others. Cosolvents, such as alcohols and others, can also be used. Various substances can also be used to enhance formulation stability, such as cyclodextrins.

The invention described herein can be combined with a therapeutic agent to more effectively manage insulin disorders, such as diabetes. This combination of single chain insulin analogue and a therapeutic agent can be used to treat a patient in need thereof. The combination can be a therapeutically effective amount of a pharmaceutically acceptable composition comprising diabetes medications.

Examples of diabetes medications include sulfonylureas, meglitinides, biguanides, thiazolidinediones, alpha-glucosidase inhibitors, or DPP-4 inhibitors. First generation sulfonylureas include Chlorpropamide (Diabinese), and second generation sulfonylureas include glipizide (Glucotrol and Glucotrol XL), glyburide (Micronase, Glynase, and Diabeta), and glimepiride (Amaryl). Meglinitides include repaglinide (Prandin) and nateglinide (Starlix). Biguanides include metformin (Glucophage). Thiazolidinediones include rosiglitazone (Avandia) and pioglitazone (ACTOS). DDP-4 inhibitors include sitagliptin (JANUVIA) and saxagliptin (ONGLYZA).

In some embodiments, compositions can be provided containing an insulin derivative and one or more additional diabetes medications that can be delivered together in a responsive manner or independently. The diabetes medication can be provided by extended release in combination with responsive release of the insulin derivative in response to increased glucose levels.

In addition to insulin and insulin analogs, other therapeutic, prophylactic or diagnostic agents can be encapsulated to treat or manage diseases or disorders. These can include small drugs, proteins or peptide, nucleic acid molecules such as DNA, mRNA and siRNA, polysaccharides, lipids, and combinations thereof. The specific therapeutic, prophylactic, or diagnostic agents encapsulated will depend upon the condition to be treated. Diagnostic agents can be released alone or in combination with therapeutic and/or prophylactic agents. Examples include radionuclides, radiopaque molecules, and MRI, x-ray or ultrasound detectable molecules.

### Dosage and Administration

In one embodiment, the composition disclosed herein can be used for clinical purpose to treat a patient. A clinical purpose includes, but is not limited to, diagnosis, prognosis, therapy, clinical trial, and clinical research. In one embodiment, the composition disclosed herein can be used for studying pharmacokinetics/pharmacodynamics.

As used herein, the term “patient” includes members of the animal kingdom including, but not limited to, human beings. As used herein, the term “mammalian host” includes members of the animal kingdom including, but not limited to, human beings. The term “mammal” is known in the art, and exemplary mammals include human, primate, bovine, porcine, canine, feline, and rodent (e.g., mice and rats).

The method of administration and use can vary depending on the disease, indication, condition or syndrome to be treated and other factors known to those in the art.

Compositions disclosed herein can be formulated for subcutaneous injection. A pharmaceutical composition formulated for injection can be formulated for sustained release. Formulations can comprise a polyethylene glycol, such as polyethylene glycol 3350, and optionally one or more additional excipients and preservatives, including excipients such as salts, polysorbate 80, sodium hydroxide or hydrochloric acid to adjust pH, and the like. Any of a number of injectable and biodegradable polymers, which can also be adhesive polymers, can be employed in a sustained release injectable formulation.

Compositions disclosed herein can be formulated for local injection, such as local injection into the intra-articular space of a joint or intramuscularly. A pharmaceutical composition formulated for local injection can be optimized for delivery via, e.g., syringe, needle, catheter, infusion pump, pen device, and the like.

Compositions disclosed herein can be formulated for administration intranasally or to the respiratory tract, e.g., inhalation, in the form of an aerosol or solution for a nebulizer, or as a microfine powder for insufflation or inhalation (e.g., topically to the lung and/or airways), alone or in combination with one or more inert carriers or additional active pharmaceutical ingredients, and in the form of a solution, a suspension, an aerosol or a dry powder formulation. A formulation for inhalation can comprise a suitable powder base, diluent or carrier substance such as lactose, glucose, dextran, mannitol or another sugar or starch. The composition can be used in any of a variety of dry powder devices, such as a reservoir dry powder inhaler, a multi-dose dry powder inhaler, or a metered dose inhaler. The composition can include additional excipients, such as an alcohol, a surfactant, a lubricant, an anti-oxidant or a stabilizing agent. Suitable propellants can include hydrocarbon, chlorofluorocarbon and hydrofluoroalkane propellants, or mixtures of any such propellants. Inhalation solutions also can be formulated in a liquefied propellant for aerosol delivery, such as with a pressurized metered dose inhaler.

For ocular applications, compositions described herein can be formulated in an ophthalmic dosage form and administered in the form of eye drops, eye washes or by means of other ocular delivery systems. Emulsions, ointments, gels, ocular inserts, biodegradable ocular inserts, liposomes, micro-particles, nanoparticles, nanospheres or ion pairing formulations can also be employed, which may, in some instances, result in increasing the ocular residence.

Compositions disclosed herein can be formulated for sublingual administration, wherein compositions herein can be in contact with the mucous membrane beneath the tongue. A composition administered sublingually, for example, can be formulated as a tablet, pill, pellet, powder, or spray. Suitable formulations may include ointments, capsules, solutions, syrups, drops, and granules.

Compositions disclosed herein can be formulated for depot administration. The depot administration can be a subcutaneous, intradermal, or intramuscular injection. The composition may be formulated as either solid or oil-based that allows for gradual absorption of the composition over a time period of hours, days, weeks, months, or years. In some aspects, suitable formulations can include zinc phosphate and PLGA polymer. In various aspects, depot formulation can be in the form of a natural or synthetic microparticle, a nanoparticle, a liposome, a carbon nanotube, a micelle, and/or a thermosensitive hydrogel. Natural or synthetic microparticles can possess high protein loading capacity and encapsulation efficiency, and can provide sustained release and controlled systemic delivery of biologically active peptides and proteins. For example, microparticles can be starch, alginate, collagen, poly (lactide-co-glycolide) (PLGA), or polycaprolactones (PCL). Nanoparticles can be colloidal carriers with sizes ranging from 10 to 1000 nm. Nanoparticles can protect the protein or peptide from degradation, prolong in vivo half-life, and provide long-term drug release. Additionally, nanoparticles can be fabricated from lipids, polymers or metal such as chitosan, alginate, PCL, polylactic acid (PLA), poly (glycolide), or PLGA. Liposomes can be bilayered vesicles with an aqueous core enclosed by phospholipid membrane of either synthetic or natural origin, and can vary in size from 20 nm to several hundred micrometers. Liposomes can protect a peptide or protein from in vivo degradation, which can prolong half-life, increase systemic circulation time, and increase bioavailability of the peptide or protein. Carbon nanotubes can be hollow cylindrical nanostructures consisting of hexagonal arrangements of sp2-hybridized carbon atoms. A peptide or protein encapsulated in a carbon nanotube can have a prolonged half-life with sustained release over time. Micelles can be nano-scaled constructs formed by self-assembly of amphiphilic molecules and comprised of an inner hydrophobic core and an outer hydrophilic corona. Micelles can encapsulate a peptide or protein in their core, which can improve chemical and physical stability of the peptide or protein. Thermosensitive hydrogels can be an alternative particulate delivery approaches. Thermosensitive hydrogels can be polymeric solutions, which undergo sol-gel phase transition to form viscoelastic gel in response to changes in temperature. They can form temperature-dependent micellular aggregates, which can undergo gelation after further temperature increment due to aggregation or packing. For delivery, a peptide or protein can be mixed with polymer in a solution state. This solution, following administration, can form an in situ gel depot at physiological temperature such that the peptide or protein can remain entrapped and this entrapped cargo can be released from the depot over a period of time, providing extended release.

Compositions disclosed herein can be formulated for administration via a pump. A composition administered via a pump can be formulated for continuous subcutaneous infusion. The pump can include the pump itself, a disposable reservoir for the composition, and a disposable infusion set, which can comprise a cannula for subcutaneous insertion under the skin and a tubing system to interface the composition reservoir to the cannula. Administration via a pump can allow for a continuous rate of administration of the composition over a period of time.

Compositions disclosed herein can be formulated for administration via an invasive device such as a stent or an implant. The implantable device can include the device itself, a reservoir for the composition, and the composition. The composition can be delivered through a reservoir in the device or as a conjugate to the implantable device. The implantable device can be a neurological implant (e.g., intraocular lens, cochlear implant, neurostimulator), a cardiovascular medical device (e.g., artificial heart valve, cardiac pacemaker, and coronary stent), an orthopaedic device (e.g., pins, rods, screws, and plates), a contraceptive device (e.g., copper- and hormone-based intrauterine devices), or a cosmetic implant (e.g., nose prosthesis, injectable filler).

Compositions disclosed herein can be advantageous compared to traditional insulin compositions. Traditionally, insulin administration involves long acting basal insulin administration and fast acting parandial insulins, which can be required after meals to lower the associated high blood glucose levels. Parandial administration can require a blood test to determine the blood glucose level, which can then be used to calculate the dose of insulin required. Typically 3 to 4 insulin injections are required per day, as well as multiple blood tests, which involve a significant degree of discomfort for patients. Additionally, minor insulin dosing changes can result in blood glucose fluctuations, which can be linked to severe cardiovascular complications. The compositions disclosed herein can respond directly to blood glucose concentrations; for example, if the activity of the insulin analog is determined by the concentration of glucose, then no steps are required to measure blood glucose and adjust the insulin dose. This can eliminate the need for multiple daily injections and can allow for dosing once daily, every second day, every three days, every four days, every five days, once weekly, once every 1.5 weeks, once every two weeks, or monthly dosing. In some instances after an injection of the pharmaceutical composition comprising the glucose-responsive insulin, an additional dose is not needed until 24 hours, 48 hours, 72 hours, 96 hours, 120 hours, 144 hours, 168 hours, or longer depending on the lifetime of the glucose-responsive insulin analog. This would significantly reduce pain and discomfort to the subject. Additionally, the glucose-responsive insulin analog can effectively function as a prandial insulin and a basal insulin, thus eliminating the need for prandial insulin and basal insulin. Compositions disclosed herein can also decrease fluctuations in blood glucose as the glucose-responsive insulin analogs are already present in the blood before meals are consumed, and thus can allow for the necessary response to changes in blood glucose levels in real time.

### Methods of Insulin Production

In various embodiments, methods and compositions described herein can utilize an expression vector to make the peptides described herein. In some aspects, by combining molecular expression technologies that employ genetically-malleable microorganisms, such as E. coli cells, to synthesize a peptide of interest with post-expression isolation and modification, one can synthesize a desired peptide rapidly and efficiently. In various embodiments, methods and compositions described herein can produce fusion peptides that can be purified using affinity separation and cleaved with a chemical reagent to release a target peptide, including a single chain insulin target peptide.

In various embodiments, methods and compositions described herein are directed to a vector that encodes an inclusion body targeting sequence, an affinity tag to facilitate purification, and a specific amino acid sequence that facilitates selective chemical cleavage. Variously, the inclusion body targeting amino acid sequence comprises from about 1 to about 125 amino acids of a ketosteroid isomerase protein or residues of oleosin, preferably residues up to residues 1-52, with or without amino acid substitutions. Such amino acid substitutions may improve chromatographic purification. The affinity tag sequence may comprise a poly-histidine, a poly-lysine, poly-aspartic acid, or poly-glutamic acid. In one embodiment, the vector further comprises an expression promoter located on the 5′ end of the affinity tag sequence. In one embodiment, methods and compositions described herein are directed to a vector that codes for a specific sequence that facilitates selective chemical cleavage to yield a peptide of interest following purification. Such chemically cleavable amino acid sequences include Trp, His-Met, or Pro-Met.

In one embodiment, methods and compositions described herein utilize a peptide expression vector, comprising: a) a first nucleotide sequence encoding an affinity tag amino acid sequence; b) a second nucleotide sequence encoding an inclusion body targeting amino acid sequence; c) a third nucleotide sequence encoding a chemically cleavable amino acid sequence; and d) a promoter in operable combination with the first, second, and third nucleotide sequences.

In one embodiment, methods and compositions described herein produce an insulin analog of commercial or therapeutic interest comprising the steps of: a) cleaving a vector with a restriction endonuclease to produce a cleaved vector; b) ligating the cleavage site to one or more nucleic acids, wherein the nucleic acids encode a desired peptide having at least a base overhang at each end configured and arranged for ligation with the cleaved vector to produce a second vector suitable for expression of a fusion peptide; c) transforming the second vector into suitable host cell; d) incubating the host cell under conditions suitable for expression of fusion peptide; e) isolation of inclusion bodies from the host cell; f) solubilization and extraction of the fusion peptide from the inclusion bodies; g) binding of the fusion peptide to a suitable affinity material; h) washing of bound fusion peptide to remove impurities; and i) cleaving the fusion peptide to release the said target SCI peptide.

An insulin analog produced by methods and compositions described herein may have significantly lower costs and/or other advantageous features. These potentially cheaper costs may lie not only in less expensive raw materials required for production, but also may lie in less chemical waste which is generated compared to the traditional process of solid phase peptide synthesis, or in more efficient processing to achieve a certain purity, thus lowering the cost of the material. Furthermore, the exclusion of a waste stream may be particularly beneficial to the environment. In various embodiments, processes according to methods and compositions described herein provide a high yield of insulin analog with high purity. In various embodiments, insulin analog produced according to methods and compositions described herein may be R&D grade or clinical grade.

### Vectors

A “promoter” can be any sequence of DNA that is active, and controls transcription in a eukaryotic cell. Preferably, the promoter is active in mammalian cells. The promoter may be constitutively expressed or inducible. Preferably, the promoter is inducible. Preferably, the promoter is inducible by an external stimulus. More preferably, the promoter is inducible by hormones or metabolites. Still more preferably, the promoter is regulatable by glucose. Even more preferably, the promoter is a pyruvate kinase gene promoter. In various embodiments, the promoter is a hepatocyte-specific L-type pyruvate kinase gene promoter.

Enhancer elements, which control transcription, can be inserted into a DNA vector construct for the production of an insulin analog, and used to enhance the expression of the target of interest.

### Inclusion-body Directing Peptides

Inclusion bodies are composed of insoluble and denatured forms of a peptide and are about 0.5-1.3 μm in diameter. These dense and porous aggregates help to simplify recombinant protein production since they have a high homogeneity of the expressed protein or peptide, result in lower degradation of the expressed protein or peptide because of a higher resistance to proteolytic attack by cellular proteases, and are easy to isolate from the rest of the cell due to differences in their density and size relative to the other cellular components. In various embodiments, the presence of inclusion bodies permits production of increased concentrations of the expressed protein or peptide due to reduced toxicity by the protein or peptide upon segregation into an inclusion body. Once isolated, the inclusion bodies may be solubilized to allow for further manipulation and/or purification.

An inclusion-body directing peptide is an amino acid sequence that helps to direct a newly translated protein or peptide into insoluble aggregates within the host cell. Prior to final isolation, in various embodiments, the target an insulin analog is produced as a fusion peptide where the fusion peptide includes as part of its sequence of amino acids an inclusion-body directing peptide. Methods and compositions described herein are applicable to a wide range of inclusion-body directing peptides as components of the expressed fusion protein or peptide.

In various embodiments, the inclusion-body directing peptide is a keto-steroid isomerase (KSI) sequence, a functional fragment thereof, or a functional homolog thereof. In various embodiments, the inclusion-body directing peptide is a BRCA-2 sequence, a functional fragment thereof, or a functional homolog thereof.

### Affinity Tag Peptides

According to methods and compositions described herein, a wide variety of affinity tags may be used. Affinity tags useful according to methods and compositions described herein may be specific for cations, anions, metals, or any other material suitable for an affinity column. In one embodiment, any peptide not possessing an affinity tag will elute through the affinity column leaving the desired fusion peptide bound to the affinity column via the affinity tag.

Specific affinity tags according to methods and compositions described herein may include poly-lysine, poly-histidine, poly-glutamic acid, or poly-arginine peptides. For example, the affinity tags may be 5-10 lysines, 5-10 histidines, 5-10 glutamic acids, or 5-10 arginines. In various embodiments, the affinity tag is a hexa-histidine sequence, hexa-lysine sequence, hexa-glutamic acid sequence, or hexa-arginine sequence. Alternatively, the HAT-tag (Clontech) may be used. In various embodiments, the affinity tag is a His-Trp Ni-affinity tag. Other tags known in the art may also be used. Examples of tags include, but are not limited to, Isopeptag, BCCP-tag, Myc-tag, Calmodulin-tag, FLAG-tag, HA-tag, MBP-tag, Nus-tag, GST-tag, GFP-tag, Thioredoxin-tag, S-tag, Softag, Streptavidin-tag, V5-tag, CBP-tag, and SBP-tag.

Without wishing to be bound by theory, it is believed that the histidine residues of a poly-histidine tag bind with high affinity to Ni-NTA or TALON resins. Both of these resins contain a divalent cation (Ni-NTA resins contain Mg2+; TALON resins contain Co2+) that forms a high affinity coordination with the His tag.

In various embodiments, the affinity tag has a pI (isoelectric point) that is at least one pH unit separate from the pI of the target insulin analog. Such difference may be either above or below the pI of the target peptide. For example, in various embodiments, the affinity tag has a pI that is at least one pH unit lower, at least two pH units lower, at least three pH units lower, at least four pH units lower, at least five pH units lower, at least six pH units lower, or at least seven pH units lower. Alternatively, the affinity tag has a pI that is at least one pH unit higher, at least two pH units higher, at least three pH units higher, at least four pH units higher, at least five pH units higher, at least six pH units higher, or at least seven pH units higher.

In various embodiments, the affinity tag is contained within the native sequence of the inclusion body directing peptide. Alternatively, the inclusion body directing peptide is modified to include an affinity tag. For example, in one embodiment, the affinity tag is a KSI, oleosin N-terminus, or BRCA2 sequence modified to include extra histidines, extra lysines, extra arginines, or extra glutamic acids.

In various embodiments, epitopes may be used such as FLAG (Eastman Kodak) or myc (Invitrogen) in conjunction with their antibody pairs.

Removal of Insulin Analog from Affinity Column Via Cleavage

Fusion peptides containing an insulin analog on the affinity column can be cleaved. In general, the cleavage step occurs by introduction of a cleavage agent which interacts with the cleavage tag of the fusion peptide resulting in cleavage of the fusion peptide and release of the target insulin analog peptide. Following cleavage, the affinity column may be flushed to elute the insulin analog target peptide while the portion of the fusion peptide containing the affinity tag remains bound to the affinity column. Following elution of the target peptide, the eluting solution may be condensed to a desired concentration. The target insulin analog peptide may be further processed and/or packaged for distribution or sale.

Control of the cleavage reaction may occur through chemical selectivity. For example, the cleavage tag may include a unique chemical moiety which is absent from the remainder of the fusion peptide such that the cleavage agent selectively interacts with the unique chemical moiety of the cleavage tag.

In various embodiments, control of the cleavage reaction can occur through a unique local environment. For example, the cleavage tag may include a chemical moiety that is present elsewhere in the fusion peptide, but the local environment differs resulting in a selective cleavage reaction at the cleavage tag. For example, in various embodiments, the cleavage tag includes a tryptophan and a charged amino acid side chain within five amino acids of the tryptophan. In various embodiments, the charged amino acid is on the amino terminus of the tryptophan amino acid.

In various embodiments, control of the cleavage reaction can occur through secondary or tertiary structure of the fusion peptide containing an insulin analog. For example, in various embodiments, where identical moieties are present in the cleavage tag and elsewhere in the fusion peptide, the other portions of the fusion peptide may fold in secondary or tertiary structure such as alpha-helices, beta-sheets, and the like, or through disulfide linkages to physically protect the susceptible moiety, resulting in selective cleavage at the cleavage tag.

In various embodiments, minor or even major differences in selectivity of the cleavage reaction for the cleavage tag over other locations in the fusion peptide can be amplified by controlling the kinetics of the cleavage reaction. For example, in various embodiments, the concentration of cleavage agent can be controlled by adjusting the flow rate of eluting solvent containing cleavage agent. In various embodiments, the concentration of cleavage agent can be maintained at a low level to amplify differences in selectivity. In various embodiments, the reservoir for receiving the eluting solvent can contain a quenching agent to stop further cleavage of target peptide that has been released from the column.

Moreover, various methods for removal of peptides from affinity columns can be excluded. For example, in some embodiments, the steps of removal can specifically exclude the step of washing an affinity column with a solution of a compound with competing affinity in the absence of a cleavage reaction. In one embodiment, the step of washing an affinity column with a solution of imidazole as a displacing agent to assist in removing a fusion peptide from an affinity column is specifically excluded. The concentration of imidazole may vary. For example, the concentration of imidazole to wash the column can include about 1-10 mM, 5-20 mM, 10-50 mM, 30-70 mM, 50-100 mM, 80-200 mM, 100-300 mM, 150-500 mM. Imidazole can be applied as a fixed concentration or as a gradient between two fixed concentration representing the lower and the upper limits. For example, a gradient of imidazole can be used to wash the column, starting from 1 mM and ending with 500 mM over a period of time.

In various embodiments, the cleavage agent can be selected from the group consisting of NBS, NCS, cyanogen bromide, Pd(H2O)4, 2-ortho iodobenzoic acid, DMSO/sulfuric acid, or a proteolytic enzyme. Various methods and cleavage agents are described in detail herein.

In one embodiment, the cleavage reaction according to methods and compositions described herein can involve the use of a mild brominating agent N-bromosuccinimde (NBS) to selectively cleave a tryptophanyl peptide bond at the amino terminus of the SCI peptide. Without wishing to be bound by theory, it is believed that in aqueous and acidic conditions, NBS can oxidize the exposed indole ring of the tryptophan side chain, thus initiating a chemical transformation that can result in cleavage of the peptide bond at this site. Accordingly, the active bromide ion halogenates the indole ring of the tryptophan residue followed by a spontaneous dehalogenation through a series of hydrolysis reactions. These reactions can lead to the formation of an oxindole derivative which promotes the cleavage reaction.

In one embodiment, the cleavage reaction according to methods and compositions described herein can involve the use of a mild oxidizing agent N-chlorosuccinimde (NCS) to selectively cleave a tryptophanyl peptide bond at the amino terminus of the target peptide. Without wishing to be bound by theory, it is believed that in aqueous and acidic conditions, NCS can oxidize the exposed indole ring of the tryptophan side chain, thus initiating a chemical transformation that can result in cleavage of the peptide bond at this site.

### EXAMPLES

The invention is further illustrated by the following non-limiting examples.

### Example 1

### Synthesis of Single Chain Insulin Analog

This example describes a method of synthesizing a single chain insulin analog. The expression vector is designed to comprise the mutation of the glucose-binding site. Ampicillin-resistant bacterial cells (E. coli) are transfected with the expression vector and are expanded overnight at 37° C. On the following day, one clone is selected to inoculate a starter culture in Luria broth with ampicillin. After 16 hrs, the starter culture is used to inoculate 1 L of Luria broth with ampicillin and cultured to an optical density of 0.5. Cells are induced with 1 mM iPTG and 0.2% L-arabinose to initiate synthesis.

Following induction, the cells are lysed in lysis buffer containing 25 mM Tris pH 8.0, 50 mM NaCl, 10% glycerol, and the protease inhibitor 1000×PMSF. High-level expression of recombinant proteins forms insoluble inclusion bodies. Insoluble inclusion bodies are collected using washing and centrifugation. Three different wash buffers are used containing varying concentrations of Tris pH 8.0, NaCl, and Triton X100. After washing out the remaining cellular components, the insoluble inclusion bodies are solubilized in a buffer containing 25 mM Tris pH 8.0, 50 mM, NaCl, 0.1 mM PMSF, and 8M urea (a chaotropic agent necessary in solubilizing protein). An acrylamide gel stained with Coomassie Blue reagent confirms synthesis and extraction of the protein.

Ni-NTA Affinity Chromatography is used to isolate the protein. The column is equilibrated with the same solubilization buffer as in the inclusion body preparation. Next, the protein is run through the resin of the column. The column is washed with five column volumes of 50% ethanol to remove urea and flow through.

Afterwards, the purified single chain insulin analog is collected. 3×NBS is loaded onto the column, which is placed on a rocker for 30 minutes. The reaction is quenched with excess N-acetylmethionine, and the flow through is collected. The column is then washed with 300 mM imidazole to discharge any remaining protein, and the flow through is collected.

### Example 2

### Binding Affinities of Allosterically Active Insulin Analog to Insulin Receptor

This example describes the binding affinities of insulin, insulin growth factor 1 (IGF1), and various insulin analogs to the insulin receptor (IR) and IGF1 receptor (IGF1R). A desirable mutant has a high binding affinity of insulin analog to the insulin receptor, as indicated by a low equilibrium dissociation constant K_{D}, and a low binding affinity to IGF1R in the presence of high levels of glucose. SEQ ID NO: 2, SEQ ID NO: 3, SEQ ID NO: 4, SEQ ID NO: 6, SEQ ID NO: 5 and SEQ ID NO: 7 are used as a starting point for designing glucose-responsive insulin analogs. Table 1 shows affinity of SEQ ID NO: 2, SEQ ID NO: 3, SEQ ID NO: 4, SEQ ID NO: 6, SEQ ID NO: 5 and SEQ ID NO: 7 for IR and IGF1-R in the absence of glucose. Computational analysis is performed on each insulin analog to predict which residues, when mutated, will result in the mutated insulin analog undergoing a conformational change that corresponds to a higher affinity for the insulin receptor when a glucose molecule is bound to the glucose binding site as compared to the mutated insulin analog affinity for the insulin receptor in the absence of a glucose molecule binding to the glucose binding site. Based on these predictions, the mutated insulin analogs are made. Normal mode analysis is then performed for each mutant insulin analog to test for whether the mutated insulin analog is a glucose-responsive insulin analog. The mutated insulin analog is considered a glucose-responsive insulin if the mutated insulin analog undergoes a conformational change upon the binding of a glucose molecule to the glucose binding site, has a low affinity for the insulin receptor when a glucose molecule is not bound to the glucose binding site, and has a higher affinity for the insulin receptor when a glucose molecule is bound to the glucose binding site.

TABLE 1
Binding Affinities of Insulin Analogs to the Insulin Receptor

| Analog | K_{D} [nM] IR | K_{D} [nM] IGF1R |
|---|---|---|
| Insulin | 0.06 | 5.66 |
| IGF1 | NA | 0.04 |
| SEQ ID NO: 2 | 0.33 | 7.52 |
| SEQ ID NO: 3 | 1.16 | 6.72 |
| SEQ ID NO: 4 | 4.48 | 12.24 |
| SEQ ID NO: 6 | 0.99 | 24.81 |
| SEQ ID NO: 5 | 4.51 | 30.38 |
| SEQ ID NO: 7 | 1.21 | 26.84 |

### Example 3

### Molecular Dynamics of an Insulin Analog

This example describes a molecular dynamics simulation of insulin and an insulin with the Wakayama mutation. Insulin with the Wakayama mutation has a valine to leucine substitution at position A3 of Insulin Chain A. Normal mode analysis was performed on each insulin. The amount of movement was calculated at each amino acid residue of the insulin B chain and displayed as a function of force. Force is proportional to distance displaced. There was an increase of movement at residue B8 of the insulin with the Wakayama mutation as compared with the insulin, as seen in FIG. 1. This indicated that the Wakayama mutation created a high transition state barrier for the conformational change from the T state to the R state at the glycine at position B8 as compared with insulin.

### Example 4

### Testing of a Single Chain Insulin Analog as a Therapeutic for Type 1 Diabetes

This example describes a method of treating insulin disorders using a glucose-responsive insulin analog. A glucose-responsive insulin analog is an SCI analog identified as having low affinity for an insulin receptor when glucose is not bound to it and high affinity for an insulin receptor when glucose is bound to it. This SCI analog is expressed recombinantly or chemically synthesized. The SCI analog is isolate and purified, and combined with a vehicle for administration to a subject. This pharmaceutical composition is to treat a subject with Type 1 Diabetes.

A dose of the pharmaceutical composition comprising the glucose-responsive insulin analog is delivered the subject via injection. A meal is eaten by the subject. Blood glucose levels are measured after 1 hour, 2 hours, 4 hours, 8 hours, 16 hours, and 24 hours. At 1 hour and 2 hours after the meal, the blood glucose level of the subject is measured to be between 100-140 mg/ml within 2 hours after a meal, and between 70 and 100 mg/ml at 4 hours, 8 hours, 16 hours, and 24 hours after the meal.

### Example 5

### Single Chain Insulin Analog as a Therapeutic for Type 1 Diabetes

This example describes a method of treating insulin disorders using a glucose-responsive insulin analog. A glucose-responsive insulin analog is an SCI analog identified as having low affinity for an insulin receptor when glucose is not bound to it and high affinity for an insulin receptor when glucose is bound to it. This SCI analog is expressed recombinantly or chemically synthesized. The SCI analog is isolate and purified, and combined with a vehicle for administration to a subject. This pharmaceutical composition is to treat a subject with Type 1 Diabetes.

A dose of the pharmaceutical composition comprising the glucose-responsive insulin analog is delivered the subject via injection. The blood glucose levels of the subject remain either between 100-140 mg/ml within 2 hours after a meal or between 70 and 100 mg/ml if more than 2 hours after a meal. An additional dose of the pharmaceutical composition comprising the glucose-response insulin analog is not needed until 24 to 168 hours later or longer depending on the lifetime of the glucose-responsive insulin analog after the previous dose.

### Example 6

### Design of a Glucose-Responsive Single Chain Insulin Analog

This example describes a method to design a glucose responsive single chain insulin analog. A single chain insulin molecule with low affinity for the insulin receptor and a high barrier (transition energy) for the T to R transition is selected. This selection is initially made by either using normal mode analysis to predict receptor affinity and transition energies for a range of sequence variants or by selecting candidates known to have these properties. Once a starting single chain insulin analog is selected, the insulin analog is mutated to comprise a glucose binding region in the linker. The mutation is chosen by comparison of the single chain insulin analog structure with the structure of glucose binding sites in known glucose binding proteins, for example glucokinase or the periplasmic glucose binding protein of Pseudomonas putida CSV86 (ppGBP). Normal mode analysis is then performed for each glucose binding single chain insulin analog in the presence or in the absence of glucose. Glucose binding single chain insulin analogs which have a lower T to R transition energy when in the presence of glucose than in the absence are selected as glucose-responsive single chain insulin analogs.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### PARAGRAPHS OF THE EMBODIMENTS

An insulin analog comprising a glucose binding site, wherein a binding of a glucose molecule to the glucose binding site changes a conformation of the insulin analog.

The insulin analog of paragraph 1, wherein the binding of the glucose molecule to the glucose binding site facilitates a conformation change of the insulin analog from a T state to an R state.

The insulin analog of paragraph 1, wherein the insulin analog has a high transition barrier for changing from a T state to an R state in an absence of the glucose molecule binding to the glucose binding site.

The insulin analog of any one of paragraphs 1-3, wherein the insulin analog has lower transition barrier for changing from a T state to an R state when the glucose molecule binds to the glucose binding site compared to an absence of the glucose molecule binding to the glucose binding site.

The insulin analog of any one of paragraphs 1-4, wherein the insulin analog has a low affinity for an insulin receptor in the absence of the glucose molecule binding to the glucose binding site.

The insulin analog of any one of paragraphs 1-5, wherein the insulin analog has a K_{D} of more than 0.1 nM, 0.5 nM, 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 25 nM, 30 nM, or 50 nM for an insulin receptor in an absence of the glucose molecule binding to the glucose binding site.

The insulin analog of any one of paragraphs 1-6, wherein the insulin analog has an increased affinity for an insulin receptor when the glucose molecule binds to the glucose binding site.

The insulin analog of any one of paragraphs 1-7, wherein the insulin analog has K_{D} of less than 0.001 nM, 0.005 nM, 0.01 nM, 0.05 nM, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2.0 nM, 2.5 nM, 3.0 nM, 5 nM, 10 nM, 15 nM, or 20 nM for an insulin receptor when the glucose molecule binds to the glucose binding site.

The insulin analog of any one of paragraphs 1-8, wherein an affinity of the insulin analog for an insulin receptor increases by at least 2-fold, 3-fold, 5-fold, 10-fold, 100-fold, 200-fold, 300-fold, or 500-fold when the glucose molecule binds to the glucose binding site as compared to in an absence of the glucose molecule binding to the glucose binding site.

The insulin analog of any one of paragraphs 1-9, wherein the glucose binding site comprises a glucokinase sequence.

The insulin analog of paragraph 10, wherein the glucokinase sequence comprises Thr 168, Lys 169, Asn 204, Asp 205, Asn 231, Glu 290, or any combination thereof.

The insulin analog of any one of paragraph 1-11, wherein the glucose binding site comprises a sequence from the periplasmic glucose binding protein of Pseudomonas putida CSV86 (ppGBP).

The insulin analog of paragraph 12, wherein the ppGBP sequence comprises His 379, Lys 92, And 301, Asp 303, Lys339, Trp 36, Glu 41, Trp 35, or any combination thereof.

The insulin analog of any one of paragraphs 1-13, wherein the glucose binding site has a K_{D} from 1 mM to 10 mM for a glucose molecule.

The insulin analog of any one of paragraph 1-14, wherein the insulin analog comprises a single chain insulin (SCI) of the formula:

B chain-C′-A chain Formula (I)
wherein the B chain and the A chain are modified human insulin chains;
wherein C′ covalently links the B chain and the A chain, and is a peptide of about 5 to 9 amino acids.

The insulin analog of paragraph 15, wherein the C′ comprises the following sequence: Y-P-G-D-X (SEQ ID NO: 1), wherein X is any amino acid.

The insulin analog of paragraph 15, wherein the C′ peptide comprises the amino acid sequence Y₁Y₂Y₃Y₄Y₅Y₆Y₇; wherein Y₁ is R or absent, Y₂ is R or absent, Y₃ is Y or V, Y₄ is P or absent, Y₅ is G or D, Y₆ is D or G, and Y₇ is any amino acid.

The insulin analog of paragraph 17, wherein Y₇ is V or K.

The insulin analog of any one of paragraphs 15-18, wherein residues within the C′ comprises the glucose binding site.

The insulin analog of any one of paragraphs 15-19, wherein residues within the B chain comprises the glucose binding site.

The insulin analog of any one of paragraphs 15-20, wherein residues within the A chain comprises the glucose binding site.

The insulin analog of any one of paragraphs 15-21, wherein residues within the C′ and B chain comprise the glucose binding site.

The insulin analog of any one of paragraphs 15-21, wherein residues within the C′ and A chain comprise the glucose binding site.

The insulin analog of any one of paragraphs 15-21, wherein residues within the C′, A chain, and B chain comprise the glucose binding site.

The insulin analog of paragraph 15, wherein the B chain peptide and the A chain peptide are linked together by two disulfide bonds.

The insulin analog of paragraph 15, wherein the B chain peptide is a peptide of 50 or fewer amino acids.

The insulin analog of paragraph 15, wherein the A chain peptide is a peptide of 50 or fewer amino acids.

The insulin analog of paragraph 15, wherein the B chain peptide is a peptide of 27-30 amino acids.

The insulin analog of paragraph 15, wherein the A chain peptide is a peptide of 21 amino acids.

The insulin analog of paragraph 15, wherein the A chain polypeptide is native human insulin B chain (SEQ ID NO: 8).

The insulin analog of paragraph 30, wherein the native human insulin A chain comprises one or more mutations at amino acid residues selected from the group consisting of Gln5, Gln15, Asn18, and Asn21.

The insulin analog of paragraph 15, wherein the B chain polypeptide is native human insulin B chain (SEQ ID NO: 9).

The insulin analog of paragraph 32, wherein the native human insulin B chain comprises one or more mutations at amino acid residues selected from the group consisting of Asn3 and Gln5.

A polynucleotide comprising a nucleic acid sequence that encodes the insulin analog according to any one of paragraphs 1-24.

A recombinant vector comprising the polynucleotide of paragraph 34.

The recombinant vector of paragraph 35, wherein the recombinant vector is a plasmid.

The recombinant vector of any one of paragraphs 35-36, comprising an inducible promoter.

The recombinant vector of any one of paragraphs 35-37, wherein the inducible promoter is IPTG.

The recombinant vector of any one of paragraphs 35-38, comprising a fusion tag.

The recombinant vector of paragraph 39, wherein the fusion tag is derived from oleosin.

The recombinant vector of any one of paragraphs 39-40, wherein the fusion tag encodes a chemically cleavable sequence.

A cell line transformed with the recombinant vector of any one of paragraphs 35-41.

A method for treating diabetes in a patient in need thereof comprising administering the insulin analog of any one of paragraphs 1-24 to the patient.

The method of paragraph 24, wherein the diabetes is type I diabetes.

A method for producing single chain insulin comprising the introduction of a recombinant vector of any one of paragraphs 35-41 into an expression system, the expression of a protein comprising a fusion tag and single chain insulin, the cleavage of said fusion tag from said single chain insulin, and isolating said single chain insulin.

A method for obtaining purified insulin analog comprising producing the insulin analog according to paragraph 45 and purifying the analog using affinity chromatography.

A therapeutic composition comprising the insulin analog of any one of paragraphs 1-24.

The therapeutic composition of paragraph 47, wherein the therapeutic composition is formulated for oral delivery.

The therapeutic composition of paragraph 47, wherein the therapeutic composition is formulated

The therapeutic composition of any one of paragraphs 47-49, wherein the therapeutic composition is formulated for once daily delivery, once weekly delivery, or once monthly delivery.

The therapeutic composition of any one of paragraphs 47-49, wherein therapeutic composition is formulated for once weekly delivery.

The therapeutic composition of any one of paragraphs 47-49, wherein therapeutic composition is formulated for delivery once every two weeks.

The therapeutic composition of any one of paragraphs 47-49, wherein therapeutic composition is formulated in a multidose form.

The therapeutic composition of any one of paragraphs 47-49, wherein therapeutic composition is formulated in a unitdose form.

A method of identifying a glucose-responsive insulin analog of any one of paragraphs 1-24, the method comprising:
i) identifying an insulin analog with a low affinity for insulin receptor;
ii) introducing at least one mutation into the insulin analog, wherein the mutation allows a glucose molecule to bind to a glucose binding site on the insulin analog or alters binding of a glucose molecule to the glucose binding site on the insulin analog;
iii) using normal mode analysis to identify the conformation of the insulin analog when a glucose molecule is bound the glucose binding site and in an absence of a glucose molecule binding to the glucose binding site;
iv) further testing for affinity of the insulin analog to the insulin receptor when a glucose molecule is bound the glucose binding site and in the absence of a glucose molecule binding to the glucose binding site; and
v) determining the insulin analog is a glucose-response insulin analog if the affinity of the insulin analog to the insulin receptor is at least 2 fold higher when a glucose molecule is bound to the glucose binding site than in the absence of a glucose molecule binding to the glucose binding site.

A method of increasing an affinity of an insulin analog to an insulin receptor, the method comprising contacting the insulin analog with a glucose molecule, wherein the glucose molecule binds to the insulin analog and thereby increases the affinity of the insulin analog towards the insulin receptor.

The method of paragraph 56, wherein the insulin analog comprises a single chain insulin (SCI) of the formula:

B chain-C′-A chain Formula (I)
wherein the B chain and the A chain are modified human insulin chains; and
wherein C′ covalently links the B chain and the A chain, and is a peptide of about 5 to 9 amino acids.

The method of paragraph 57, wherein the C′ comprises the following sequence: Y-P-G-D-X (SEQ ID NO: 1), wherein X is any amino acid.

The method of paragraph 57, wherein the C′ peptide comprises the amino acid sequence Y₁Y₂Y₃Y₄Y₅Y₆Y₇; wherein Y₁ is R or absent, Y₂ is R or absent, Y₃ is Y or V, Y₄ is P or absent, Y₅ is G or D, Y₆ is D or G, and Y₇ is any amino acid.

The method of paragraph 59, wherein Y₇ is V or K.

The method of any one of paragraphs 57-60, wherein residues within the C′ comprises the glucose binding site.

The method of any one of paragraphs 57-61, wherein residues within the B chain comprises the glucose binding site.

The method of any one of paragraphs 57-62, wherein residues within the A chain comprises the glucose binding site.

The method of any one of paragraphs 57-63, wherein residues within the C′ and B chain comprise the glucose binding site.

The method of any one of paragraphs 57-63, wherein residues within the C′ and A chain comprise the glucose binding site.

The method of any one of paragraphs 57-65, wherein residues within the C′, A chain, and B chain comprise the glucose binding site.

The method of paragraph 57, wherein the B chain peptide and the A chain peptide are linked together by two disulfide bonds.

The method of paragraph 57, wherein the B chain peptide is a peptide of 50 or fewer amino acids.

The method of paragraph 57, wherein the A chain peptide is a peptide of 50 or fewer amino acids.

The method of paragraph 57, wherein the B chain peptide is a peptide of 27-30 amino acids.

The method of paragraph 57, wherein the A chain peptide is a peptide of 21 amino acids.

The insulin analog of paragraph 57, wherein the A chain polypeptide is native human insulin B chain (SEQ ID NO: 8).

The insulin analog of paragraph 72, wherein the native human insulin A chain comprises one or more mutations at amino acid residues selected from the group consisting of Gln5, Gln15, Asn18, and Asn21.

The insulin analog of paragraph 57, wherein the B chain polypeptide is native human insulin B chain (SEQ ID NO: 9).

The insulin analog of paragraph 74, wherein the native human insulin B chain comprises one or more mutations at amino acid residues selected from the group consisting of Asn3 and Gln5.

The method of any one of paragraphs 57-71, wherein an affinity of the insulin analog for an insulin receptor increases by at least 2-fold, 3-fold, 5-fold, 10-fold, 100-fold, 200-fold, 300-fold, or 500-fold when the glucose molecule binds to the glucose binding site as compared to in an absence of the glucose molecule binding to the glucose binding site.

1. An insulin analog comprising a glucose binding site, wherein a binding of a glucose molecule to the glucose binding site changes a conformation of the insulin analog.

2. The insulin analog of claim 1, wherein the binding of the glucose molecule to the glucose binding site facilitates a conformation change of the insulin analog from a T state to an R state.

3. The insulin analog of claim 1, wherein the insulin analog has a high transition barrier for changing from a T state to an R state in an absence of the glucose molecule binding to the glucose binding site.

4. A method of increasing an affinity of an insulin analog to an insulin receptor, the method comprising contacting the insulin analog with a glucose molecule, wherein the glucose molecule binds to the insulin analog and thereby increases the affinity of the insulin analog towards the insulin receptor.

5. The method of claim 4, wherein the insulin analog comprises a single chain insulin (SCI) of the formula:
B chain-C′-A chain Formula (I)
wherein the B chain and the A chain are modified human insulin chains; and
wherein C′ covalently links the B chain and the A chain, and is a peptide of about 5 to 9 amino acids.

6. The method of claim 5, wherein the C′ comprises the following sequence: Y-P-G-D-X (SEQ ID NO: 1), wherein X is any amino acid.

7. The method of claim 5, wherein the C′ peptide comprises the amino acid sequence Y₁Y₂Y₃Y₄Y₅Y₆Y₇; wherein Y₁ is R or absent, Y₂ is R or absent, Y₃ is Y or V, Y₄ is P or absent, Y₅ is G or D, Y₆ is D or G, and Y₇ is any amino acid.

8. The method of claim 7, wherein Y₇ is V or K.

9. The method of claim 5, wherein the B chain peptide and the A chain peptide are linked together by two disulfide bonds.

10. The method of claim 5, wherein the B chain peptide is a peptide of 50 or fewer amino acids.

11. The method of claim 5, wherein the A chain peptide is a peptide of 50 or fewer amino acids.

12. The method of claim 5, wherein the B chain peptide is a peptide of 27-30 amino acids.

13. The method of claim 5, wherein the A chain peptide is a peptide of 21 amino acids.

14. The method of claim 5, wherein the B chain polypeptide of the insulin analog comprises native human insulin B chain (SEQ ID NO: 8).

15. The method of claim 14, wherein the native human insulin A chain comprises one or more mutations at amino acid residues selected from the group consisting of Gln5, Gln15, Asn18, and Asn21.

16. The method of claim 5, wherein the B chain polypeptide is native human insulin B chain (SEQ ID NO: 9).

17. The method of claim 16, wherein the native human insulin B chain comprises one or more mutations at amino acid residues selected from the group consisting of Asn3 and Gln5.

18. The method of claim 5, wherein the glucose binding site is comprised by residues within the C′ chain, the B chain, the A chain, or any combination thereof.


SEQUENCE LISTING (32 sequences)

SEQ ID NO: 1 - protein, 5 aa, Artificial Sequence
  Description of Artificial Sequence Synthetic peptide
  MOD_RES (5)..(5): Any amino acid
YPGDX

SEQ ID NO: 2 - protein, 58 aa, Artificial Sequence
  Description of Artificial Sequence Synthetic polypeptide
FVNQHLCGSHLVEALYLVCGERGFFYTPKTRRYPGDVGIVEQCCTSICSLYQLENYCG

SEQ ID NO: 3 - protein, 56 aa, Artificial Sequence
  Description of Artificial Sequence Synthetic polypeptide
FVNQHLCGSHLVEALYLVCGERGFFYTPKTRVDGVGIVEQCCTSICSLYQLENYCG

SEQ ID NO: 4 - protein, 55 aa, Artificial Sequence
  Description of Artificial Sequence Synthetic polypeptide
FVNQHLCGSHLVEALYLVCGERGFFYTPKTVDGKGIVEQCCTSICSLYQLENYCG

SEQ ID NO: 5 - protein, 55 aa, Artificial Sequence
  Description of Artificial Sequence Synthetic polypeptide
FVDQHLCGSHLVEALYLVCGERGFFYTPKTYDGKGIVEQCCTSICSLYQLENYCG

SEQ ID NO: 6 - protein, 55 aa, Artificial Sequence
  Description of Artificial Sequence Synthetic polypeptide
FVNQHLCGSHLVEALYLVCGERGFFYTPKTYDGKGIVEQCCTSICSLYQLENYCG

SEQ ID NO: 7 - protein, 55 aa, Artificial Sequence
  Description of Artificial Sequence Synthetic polypeptide
FVGQHLCGSHLVEALYLVCGERGFFYTPKTYDGKGIVEQCCTSICSLYQLENYCG

SEQ ID NO: 8 - protein, 21 aa, Homo sapiens
GIVEQCCTSICSLYQLENYCN

SEQ ID NO: 9 - protein, 30 aa, Homo sapiens
FVNQHLCGSHLVEALYLVCGERGFFYTPKT

SEQ ID NO: 10 - protein, 58 aa, Artificial Sequence
  Description of Artificial Sequence Synthetic polypeptide
  MOD_RES (1)..(1): F or absent
  MOD_RES (2)..(2): V or absent
  MOD_RES (3)..(3): N, A, K, D, or absent
  MOD_RES (31)..(31): R or absent
  MOD_RES (32)..(32): R or absent
  MOD_RES (33)..(33): Y or V
  MOD_RES (34)..(34): P or absent
  MOD_RES (35)..(35): G or D
  MOD_RES (36)..(36): D or G
  MOD_RES (37)..(37): V or K
  MOD_RES (58)..(58): N, G, A, D, or Q
XXXQHLCGSHLVEALYLVCGERGFFYTPKTXXXXXXXGIVEQCCTSICSLYQLENYCX

SEQ ID NO: 11 - protein, 7 aa, Artificial Sequence
  Description of Artificial Sequence Synthetic peptide
  MOD_RES (1)..(1): R or absent
  MOD_RES (2)..(2): R or absent
  MOD_RES (3)..(3): Y or V
  MOD_RES (4)..(4): P or absent
  MOD_RES (5)..(5): G or D
  MOD_RES (6)..(6): D or G
  MOD_RES (7)..(7): V or K
XXXXXXX

SEQ ID NO: 12 - protein, 7 aa, Artificial Sequence
  Description of Artificial Sequence Synthetic peptide
  MOD_RES (1)..(1): R or absent
  MOD_RES (2)..(2): R or absent
  MOD_RES (3)..(3): Y or V
  MOD_RES (4)..(4): P or absent
  MOD_RES (7)..(7): V or K
XXXXGDX

SEQ ID NO: 13 - protein, 7 aa, Artificial Sequence
  Description of Artificial Sequence Synthetic peptide
  MOD_RES (1)..(1): R or absent
  MOD_RES (2)..(2): R or absent
  MOD_RES (3)..(3): Y or V
  MOD_RES (4)..(4): P or absent
  MOD_RES (7)..(7): V or K
XXXXDGX

SEQ ID NO: 14 - protein, 7 aa, Artificial Sequence
  Description of Artificial Sequence Synthetic peptide
  MOD_RES (1)..(1): R or absent
  MOD_RES (2)..(2): R or absent
  MOD_RES (3)..(3): Y or V
  MOD_RES (4)..(4): P or absent
XXXXDGV

SEQ ID NO: 15 - protein, 7 aa, Artificial Sequence
  Description of Artificial Sequence Synthetic peptide
  MOD_RES (1)..(1): R or absent
  MOD_RES (2)..(2): R or absent
  MOD_RES (3)..(3): Y or V
  MOD_RES (4)..(4): P or absent
XXXXDGK

SEQ ID NO: 16 - protein, 7 aa, Artificial Sequence
  Description of Artificial Sequence Synthetic peptide
RRYPGDV

SEQ ID NO: 17 - protein, 7 aa, Artificial Sequence
  Description of Artificial Sequence Synthetic peptide
RRYPDGV

SEQ ID NO: 18 - protein, 6 aa, Artificial Sequence
  Description of Artificial Sequence Synthetic peptide
RRVDGV

SEQ ID NO: 19 - protein, 6 aa, Artificial Sequence
  Description of Artificial Sequence Synthetic peptide
RYPDGV

SEQ ID NO: 20 - protein, 5 aa, Artificial Sequence
  Description of Artificial Sequence Synthetic peptide
RVDGV

SEQ ID NO: 21 - protein, 4 aa, Artificial Sequence
  Description of Artificial Sequence Synthetic peptide
VDGK

SEQ ID NO: 22 - protein, 4 aa, Artificial Sequence
  Description of Artificial Sequence Synthetic peptide
YDGK

SEQ ID NO: 23 - protein, 10 aa, Artificial Sequence
  Description of Artificial Sequence Synthetic peptide
  MISC_FEATURE (1)..(10): This sequence may encompass 5-10 residues
KKKKKKKKKK

SEQ ID NO: 24 - protein, 10 aa, Artificial Sequence
  Description of Artificial Sequence Synthetic peptide
  MISC_FEATURE (1)..(10): This sequence may encompass 5-10 residues
HHHHHHHHHH

SEQ ID NO: 25 - protein, 10 aa, Artificial Sequence
  Description of Artificial Sequence Synthetic peptide
  MISC_FEATURE (1)..(10): This sequence may encompass 5-10 residues
EEEEEEEEEE

SEQ ID NO: 26 - protein, 10 aa, Artificial Sequence
  Description of Artificial Sequence Synthetic peptide
  MISC_FEATURE (1)..(10): This sequence may encompass 5-10 residues
RRRRRRRRRR

SEQ ID NO: 27 - protein, 6 aa, Artificial Sequence
  Description of Artificial Sequence Synthetic peptide
HHHHHH

SEQ ID NO: 28 - protein, 6 aa, Artificial Sequence
  Description of Artificial Sequence Synthetic peptide
KKKKKK

SEQ ID NO: 29 - protein, 6 aa, Artificial Sequence
  Description of Artificial Sequence Synthetic peptide
EEEEEE

SEQ ID NO: 30 - protein, 6 aa, Artificial Sequence
  Description of Artificial Sequence Synthetic peptide
RRRRRR

SEQ ID NO: 31 - protein, 7 aa, Artificial Sequence
  Description of Artificial Sequence Synthetic peptide
  MOD_RES (1)..(1): R or absent
  MOD_RES (2)..(2): R or absent
  MOD_RES (3)..(3): Y or V
  MOD_RES (4)..(4): P or absent
  MOD_RES (5)..(5): G or D
  MOD_RES (6)..(6): D or G
  MOD_RES (7)..(7): Any amino acid
XXXXXXX

SEQ ID NO: 32 - protein, 58 aa, Artificial Sequence; identical to SEQ ID NO: 10
  Description of Artificial Sequence Synthetic polypeptide
  MOD_RES (1)..(1): F or absent
  MOD_RES (2)..(2): V or absent
  MOD_RES (3)..(3): N, A, K, D, or absent
  MOD_RES (31)..(31): R or absent
  MOD_RES (32)..(32): R or absent
  MOD_RES (33)..(33): Y, R, or absent
  MOD_RES (34)..(34): P, V or Y
  MOD_RES (35)..(35): G or D
  MOD_RES (36)..(36): D or G
  MOD_RES (37)..(37): V or K
  MOD_RES (58)..(58): N, G, A, D, or Q